(19) United States
(12) Patent Application Publication
(10) Pub. No.: US 2025/0114518 A1
(43) Pub. Date: Apr. 10, 2025

(54) APPARATUS FOR INFUSING MEDICAL LIQUID WITH MAGNETIC COUPLING FOR DISPOSABLE MODULE AND REUSABLE MODULE

(71) Applicant: CureStream Co.,Ltd, Daegu (KR)

(72) Inventor: Dae Jong PARK, Seoul (KR)

(73) Assignee: CureStream Co.,Ltd, Daegu (KR)

(21) Appl. No.: 18/906,954

(22) Filed: Oct. 4, 2024

(30) Foreign Application Priority Data
Oct. 6, 2023 (KR) 10-2023-0133716

(51) Int. Cl. A61M 5/142; A61M 5/14; A61M 39/24

(52) U.S. Cl. CPC A61M 5/14216; A61M 5/1413; A61M 39/24; A61M 2202/0007; A61M 2202/04; A61M 2205/106; A61M 2205/121; A61M 2205/123

(57) ABSTRACT

The present invention relates to a liquid infusion device composed of a combination of a reusable module, which can be used repeatedly, and a disposable module, which is replaced after use. The reusable module (100) includes a solenoid (130) that generates magnetic force when powered, a first movable body (140) that performs a reciprocating motion driven by the magnetic force from the solenoid, and a second movable body (150) that moves in conjunction with the reciprocating motion of the first movable body. The disposable module (200) consists of a reservoir (230) for storing the liquid and a piston-check valve module (250) that receives the liquid stored in the reservoir and discharges it externally. When the reusable module and the disposable module are coupled, the piston-check valve module is connected to the second movable body through the attractive force of a magnet.

### CROSS-REFERENCE TO RELATED APPLICATION

This application claims priority to Korean Patent Application No. 10-2023-0133716, filed in the Korean Intellectual Property Office on Oct. 6, 2023, the entire contents of which are hereby incorporated by reference.

### TECHNICAL FIELD

The present invention relates to a medical liquid infusion device composed of a reusable module and a disposable module, allowing the disposable module to be replaced for use. Specifically, it pertains to a liquid infusion device in which the lower coupling part of a piston, which performs a reciprocating motion to discharge the liquid, is coupled using a magnet at the joint between the reusable module and the disposable module.

### BACKGROUND ART

Various liquid infusion devices for injecting medical liquids such as hormones into the human body for the treatment of various diseases are widely used. For example, an insulin infusion device is used to inject liquid into a patient's body for diabetes treatment. While such infusion devices may be used by medical professionals such as doctors or nurses, in most cases, they are operated by patients themselves or by caregivers. In the case of diabetic patients, particularly pediatric diabetic patients, it is necessary to inject liquids such as insulin into the body at regular intervals. Therefore, patch-type liquid infusion devices, which can be attached to the body for a certain period, have been developed and can be used in a state where they are attached to the patient's body for a specific period. An insulin pump is a medical device used by individuals with diabetes to help control their blood sugar levels. The device delivers insulin continuously into the body to help maintain a consistent blood glucose level. It usually works by delivering insulin through a catheter placed under the skin, and it is commonly used by people with type 1 diabetes. Unlike single-use injections, an insulin pump provides a continuous supply of insulin, allowing for more precise blood sugar control. Some pumps can even connect with glucose monitors to automatically adjust insulin delivery based on blood sugar readings. This device improves convenience for users and is especially helpful for individuals who find it difficult to time their insulin injections correctly throughout the day.

It is important for such liquid infusion devices to inject the medical liquid in precise quantities. In order to inject the liquid in a measured amount, the device needs to have an internal structure that distributes the liquid accurately.

### SUMMARY

The present invention aims to provide a structure that allows for more precise control of small amounts of liquid injected into the human body. Additionally, the invention proposes a design in which the device's configuration can be separated into a reusable module and a disposable module, and these parts are coupled using a magnet to drive the piston for liquid discharge. This simple structure enhances the reliability of the coupling.

The present invention relates to a liquid infusion device composed of a combination of a reusable module, which can be used repeatedly, and a disposable module, which is replaced after use.

The reusable module 100 includes a solenoid 130 that generates magnetic force when powered, a first movable body 140 that performs a reciprocating motion through the magnetic force generated by the solenoid, and a second movable body 150 that reciprocates in conjunction with the first movable body's motion. The disposable module 200 consists of a reservoir 230 that stores the liquid, and a piston-check valve module 250 that receives the liquid stored in the reservoir and discharges it to the outside. When the reusable module and disposable module are coupled, the piston-check valve module is connected to the second movable body through the attractive force of a magnet.

The disposable module 200 further includes a piston lower member 256, which is formed with an increased radius at the lower part of the piston 255, and a coupling magnet M provided in the piston lower member 256. The upper side of the second movable body 150 is equipped with a top coupling member made of metal, which interacts with the attractive force of the coupling magnet.

The top coupling member may be composed of a single part, or it may consist of a central coupling member 157 located at the center and an outer coupling member 158 located on the outer side.

The disposable module is equipped with a cylindrical SUS pipe 257 that encloses the outer circumferential surface of the piston lower member 256. The upper inner surface of the SUS pipe is where the piston lower member is inserted and coupled, and the lower part of the SUS pipe 257 is where the top coupling member is inserted and coupled to the coupling magnet M.

When the reusable module and disposable module are coupled, it is preferable that the lower end of the SUS pipe is inserted into the inside of the reusable module for coupling.

Additionally, the present invention further includes a semicircular metal plate 275 provided in the disposable module and a plurality of pogo pins 175 provided in the reusable module, which are formed at the contact point with the metal plate. When the reusable module and disposable module are coupled, two of the pogo pins electrically contact each other through the metal plate, thereby performing a wake-up function. The pogo pins can also be used as charging terminals when the reusable module is in a separated state.

In the liquid infusion device of the present invention, the piston, which plays a key role in discharging the liquid, is coupled in a simple structure that enhances the reliability and accuracy of the connection. This structure ensures that the liquid infusion operation is carried out smoothly.

### BRIEF DESCRIPTION OF THE DRAWINGS

The above and other objects, features and advantages of the present disclosure will be described with reference to the accompanying drawings described below, where similar reference numerals indicate similar elements, but not limited thereto, in which:

FIGS. 1 and 2 show the front and rear views of the liquid infusion device according to the present invention.

FIG. 3 shows the separated state of the reusable module and the disposable module in theliquid infusion device according to the present invention.

FIG. 4 is an exploded perspective view of the reusable module in the liquid infusion device according to the present invention.

FIG. 5 is an exploded perspective view of the disposable module in the liquid infusion device according to the present invention.

FIGS. 6 to 9 show the internal structure and operation of the piston check valve module in the liquid infusion device according to the present invention.

FIGS. 10 to 12 show the overall appearance and internal structure of the disposable module in the liquid infusion device according to the present invention.

FIGS. 13 to 14 show the overall appearance and internal structure of the reusable module in the liquid infusion device according to the present invention.

FIG. 15 shows the cross-sectional view of a specific part where the reusable module and disposable module are coupled in the liquid infusion device according to the present invention.

### DETAILED DESCRIPTION

The objectives, specific advantages and novel features of the present disclosure will become more apparent from the following detailed description and the preferred embodiments, which are associated with the accompanying drawings. In addition, terms described herein are terms defined in consideration of functions in the present invention, which may vary according to the intention or convention of a user or an operator. Therefore, definitions of these terms should be made based on the contents throughout the present specification.

Hereinafter, the present disclosure will be described in detail with reference to the accompanying drawings.

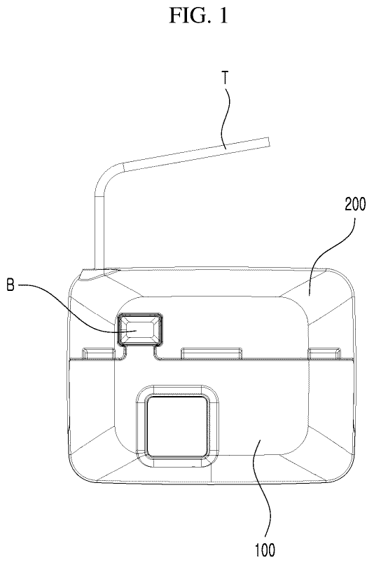

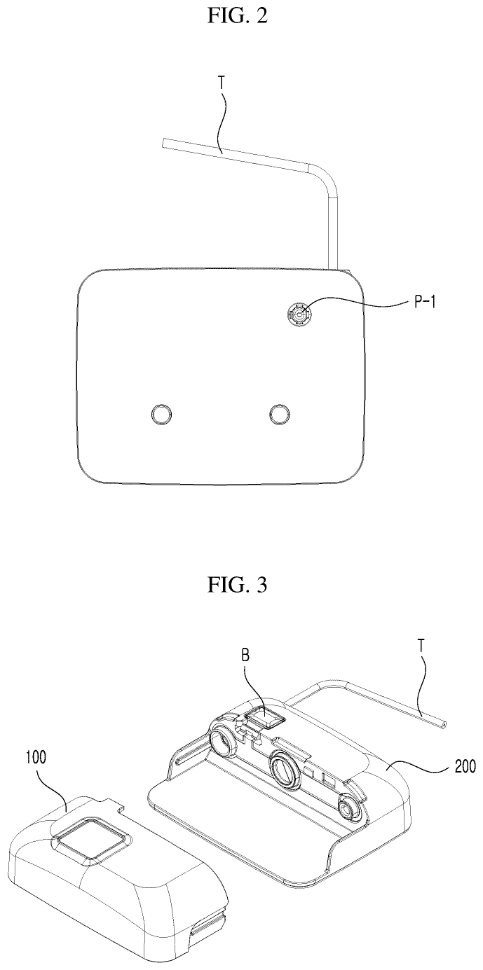

Referring to FIGS. 1 to 3, the liquid infusion device according to one embodiment of the present invention consists of a reusable module (a rechargeable module) 100, which can be charged and reused, and a disposable module 200, which is replaced after use. It further includes an attachment member (not shown) that attaches the disposable module to the body and a tube T that guides the liquid discharged from the disposable module to a needle (not shown) inserted into the body. Additionally, the liquid infusion device is equipped with a release button B on the front, which allows the reusable module and disposable module to be separated. By pressing this release button, the modules can be detached from each other (FIG. 3).

### [Structure of the Reusable Module and Disposable Module]

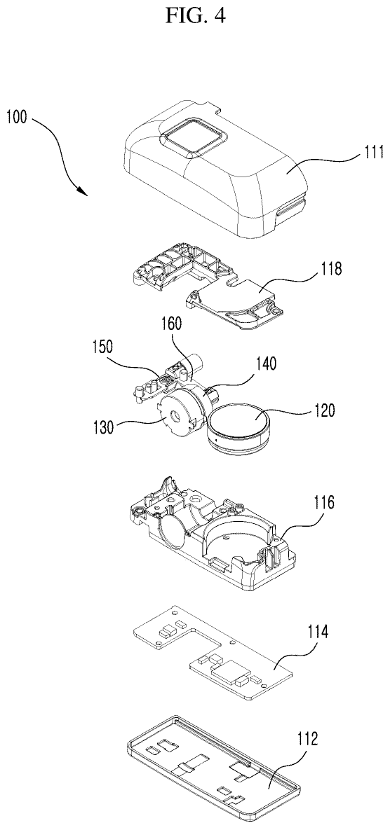

Referring to FIG. 4, the reusable module 100 is designed to be separable from the disposable module 200 using the release button B and can be reused repeatedly. The reusable module 100 includes an upper case 111 and a lower case 112, and inside these cases are a main PCB 114, a lower bracket 116, and an upper bracket 118. The components housed by the upper and lower brackets include a battery 120 that stores power, a solenoid 130 that generates magnetic force powered by the battery, a first movable body 140 that reciprocates up and down by the magnetic force generated by the solenoid, and a second movable body 150 that operates in conjunction with the reciprocating motion of the first movable body to discharge the liquid stored in the disposable module 200. Additionally, a lever 160 is provided to link the up-and-down motions of the first and second movable bodies.

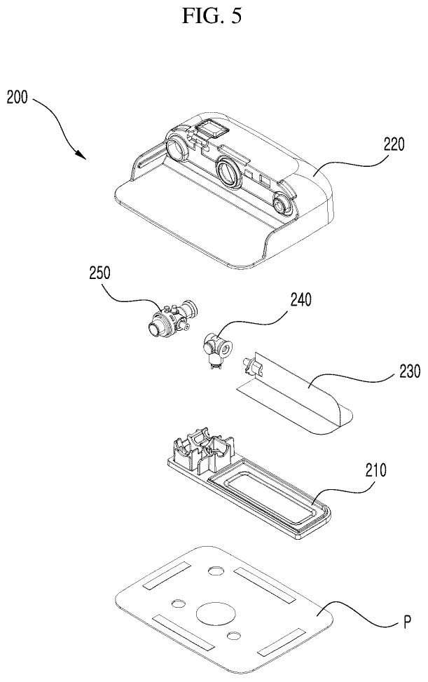

FIG. 5 illustrates the disposable module 200 of the liquid infusion device according to the present invention. The disposable module 200 includes an inner case 210 and an outer case 220, which is coupled on top of the inner case. Inside these cases are components such as a reservoir 230 for storing the liquid, a piston-check valve module 250 for discharging a set amount of liquid, and a connector 240 that connects the reservoir and the piston-check valve module to deliver the liquid. The liquid stored in the reservoir 230 is transferred to the piston-check valve module 250 via the connector 240.

### [Quantitative Control Structure]

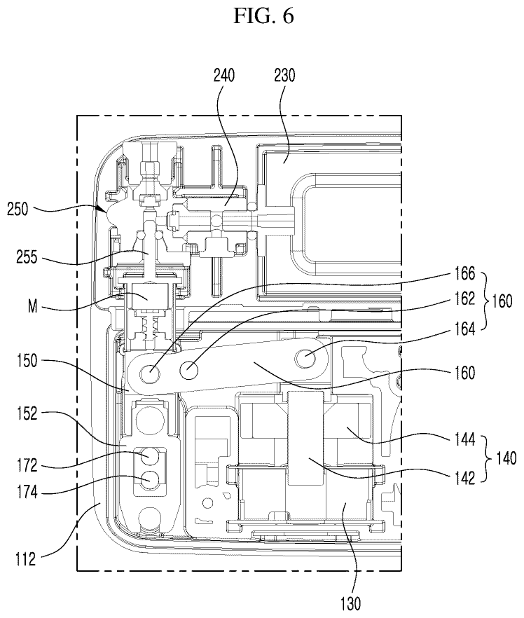

The following will explain the structure for discharging the liquid, referring to FIGS. 6 to 9, in the state where the reusable module 100 and the disposable module 200 are combined. The first movable body 140 of the reusable module 100 has a moving shaft 142 inside and an outer magnet 144 around the outer circumference of the moving shaft. The moving shaft 142 moves up and down through the attraction and repulsion applied to the outer magnet 144 by the magnetic force generated by the solenoid 130. The lever 160 is arranged in an approximately horizontal direction and is a component in which both ends move up and down around a pivot axis 162 located at a specific point between the two ends.

In the context of the up-and-down reciprocation described here, ‘upward and downward direction’ refers to the upward and downward movement as seen in FIG. 6, which is parallel to the longitudinal direction of the moving shaft 142. The upper end of the moving shaft 142 of the first movable body 140 is connected to one end 164 of the lever 160, and the other end 166 of the lever 160 is connected to the second movable body 150. The pivot axis 162 may be positioned closer to the other end rather than the exact center between the two ends, and in this invention, the pivot axis 162 is located closer to the second movable body.

By positioning the pivot axis closer to the second movable body, it allows for finer control of small amounts of liquid, and the second movable body can perform its reciprocating motion with relatively less force from the solenoid. In other words, the up-and-down reciprocating distance of the second movable body is smaller than that of the first movable body, allowing for more precise control of the small amount of liquid discharged from the quantitative control space 251, which will be explained below. Additionally, due to the leverage principle, the second movable body can be operated with a smaller force than the force required for the up-and-down movement of the first movable body.

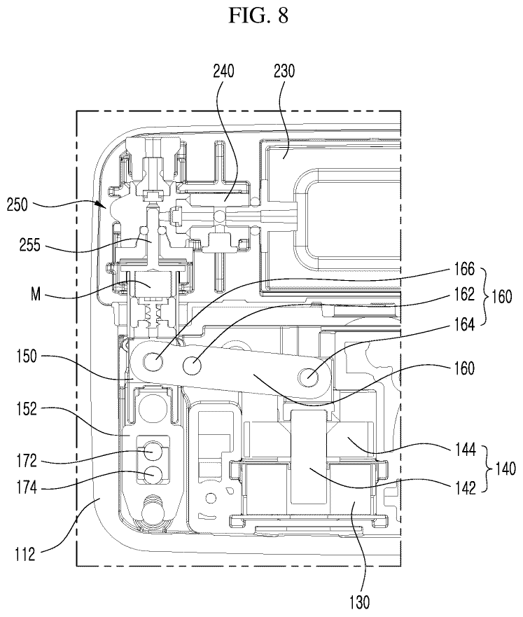

When the first movable body 140 moves forward (upward), the second movable body 150 moves backward (downward) (as shown in FIG. 6), and when the first movable body 140 moves backward (downward), the second movable body 150 moves forward (upward) (as shown in FIG. 8).

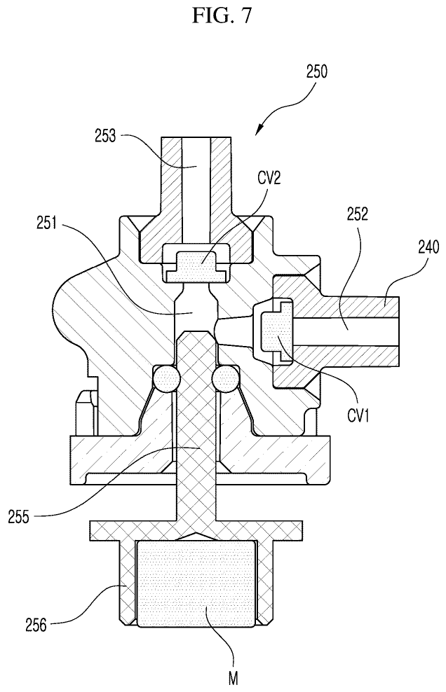

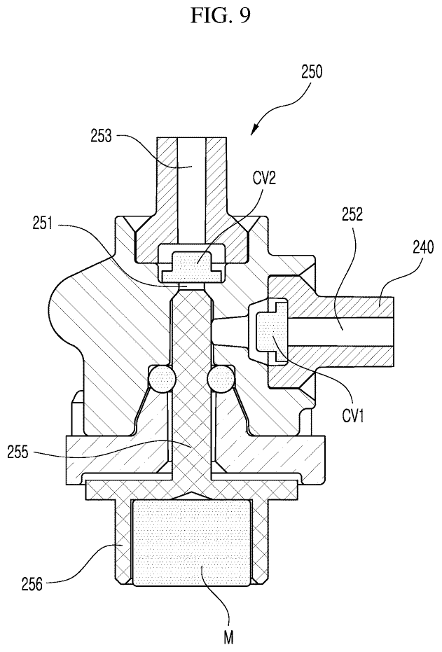

FIGS. 6 and 8 show the overall movement of the first and second movable bodies, while FIGS. 7 and 9 are enlarged detailed views that explain the operation of the piston-check valve module 250 in the disposable module 200, as shown in FIGS. 6 and 8.

The structure of the piston-check valve module 250 in the disposable module 200 is explained. The piston-check valve module 250 contains a quantitative control space 251, which is a space where a certain amount of liquid is supplied and stored. A supply passage 252 for injecting the liquid is formed laterally to the quantitative control space 251, and a discharge passage 253 for discharging the liquid is formed above the quantitative control space 251. The quantitative control space 251 is a cylindrical space formed in the vertical direction, and the piston 255 moves up and down within this space. A circular piston lower member 256, which is formed with an increased radius, is provided at the lower part of the piston 255.

The piston lower member 256 is coupled to the top of the second movable body 150 of the reusable module 100, and together with the second movable body, it performs an up-and-down reciprocating motion, causing the piston to reciprocate up and down. The piston lower member 256 is attached to the top of the second movable body 150 of the reusable module 100 through a coupling magnet M located inside it. When the first movable body 140 of the reusable module 100 moves up and down due to the operation of the solenoid 130, the second movable body 150 reciprocates accordingly, resulting in the piston 255 moving up and down within the quantitative control space 251.

When the piston 255 moves downward, the quantitative control space 251 opens, allowing the infusion process to take place as the liquid stored in the reservoir 230 is injected into the quantitative control space through the supply passage 252 (FIGS. 6 and 7). Then, when the piston 255 moves upward, the liquid in the quantitative control space 251 is discharged through the discharge passage 253 and delivered to the tube (T in FIG. 1), where it is supplied into the human body, completing the discharge process (FIGS. 8 and 9). The piston also functions as a check valve during the liquid discharge process. When the liquid is discharged from the quantitative control space, the piston 255 closes the supply passage 252 on its side outer surface, preventing the liquid from flowing back into the reservoir through the supply passage 252, thus acting as a type of check valve.

Both the supply passage 252, through which the liquid is injected into the quantitative control space 251, and the discharge passage 253, through which the liquid is discharged from the quantitative control space 251, are equipped with the first and second check valves CV1, CV2 to block reverse flow. The first check valve CV1 allows the liquid to flow only from the connector 240 into the quantitative control space 251 and prevents movement in the opposite direction, while the second check valve CV2 allows the liquid to flow only from the quantitative control space 251 toward the tube and prevents movement in the opposite direction.

The present invention, as described above, involves the construction of two check valves arranged perpendicular to each other in the supply passage and discharge passage, along with a quantitative control space utilizing a piston. These components are integrated into a single unified module, significantly reducing the product size and developing a compact structure.

By incorporating the structure of the piston-check valve module 250 in this invention, a consistent small amount of liquid is always injected into the quantitative control space 251, ensuring that it can be delivered to the human body. In other words, with a single up-and-down reciprocating motion of the piston 255, the amount of liquid corresponding to the quantitative control space is injected into the body. The specific amount of injected liquid can be determined by the size of the quantitative control space 251 (such as the diameter of the piston 255, the stroke length of the piston, etc.), and this can be adjusted based on the specific circumstances of liquid delivery.

### [Structure of the Coupling Between the Second Movable Body and the Piston]

The liquid infusion device of the present invention is composed of a combination of a reusable module 100, which is used repeatedly, and a disposable module 200, which is replaced after use. The front release button (B in FIG. 1) is used to separate and couple the outer cases of the reusable and disposable modules. When the reusable module 100 and the disposable module 200 are combined, as shown in FIG. 6, the piston-check valve module 250 of the disposable module 200 and the second movable body 150 are coupled in the vertical direction, allowing the piston 255 of the piston-check valve module to reciprocate up and down through the vertical reciprocating motion of the second movable body. To achieve this, the piston 255 and the second movable body 150 must be coupled, and the piston lower member 256 is designed to connect with the top of the second movable body 150. In this invention, the coupling is achieved using a magnet (M in FIG. 6).

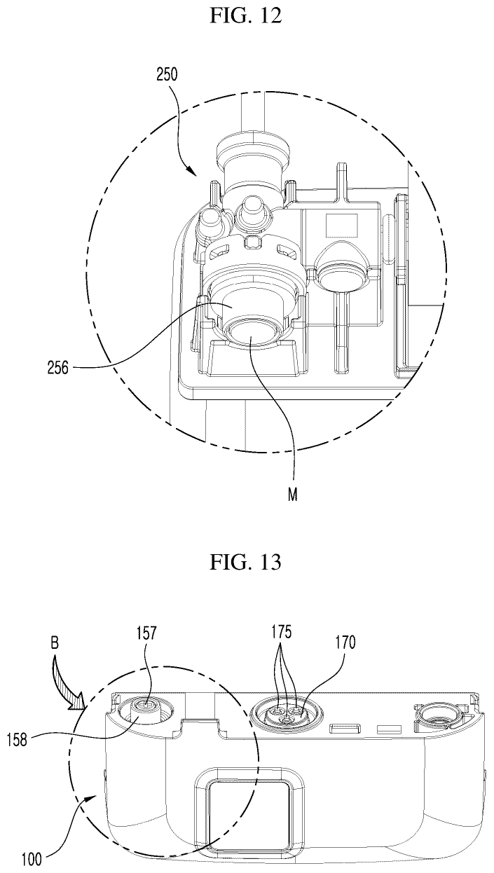

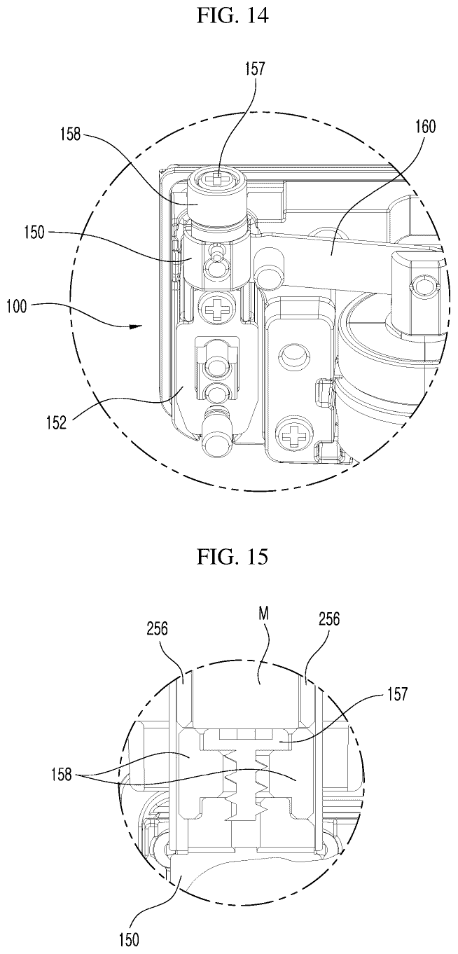

FIGS. 10 to 12 show the external appearance of the disposable module 200 of the present invention, along with the internal structure excluding certain components. FIGS. 13 and 14 illustrate the external appearance of the reusable module 100 of the present invention, also excluding some internal components. In FIG. 10, the section marked ‘A’ indicates the piston-check valve module 250 of the disposable module 200, while in FIG. 13, the section marked ‘B’ indicates the part corresponding to the second movable body of the reusable module 100. During the coupling process of the disposable module 200 and the reusable module 100, section A and section B are vertically aligned and coupled together, allowing the vertical reciprocating motion of the second movable body 150 to be transferred to the piston 255. FIG. 15 is an enlarged cross-sectional view showing the part where sections A and B are coupled together.

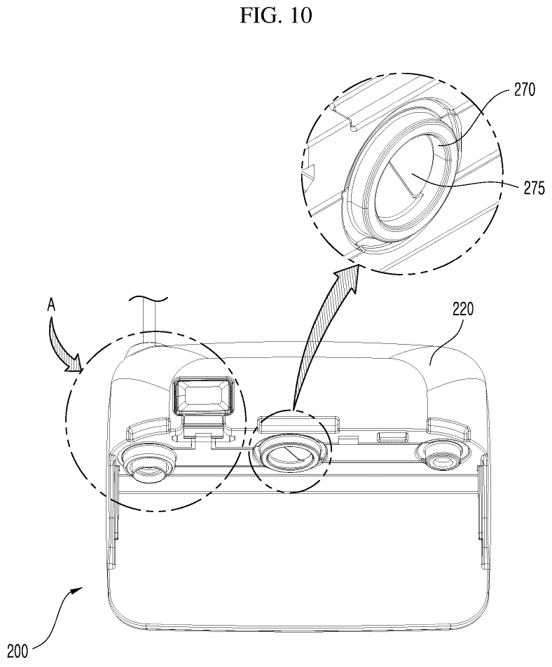

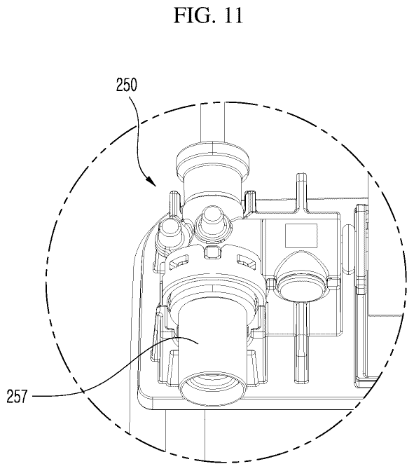

Referring to FIGS. 11 and 12, in the disposable module 200, the lower part of the piston 255 in the piston-check valve module 250 is equipped with a circular piston lower member 256, which is formed with an increased radius. The piston lower member 256 is open at the bottom, with a groove formed inside, and the piston 255 is attached at the top. A coupling magnet (M) is provided in the groove formed inside. The piston lower member 256 is surrounded and coupled by a cylindrical SUS pipe 257. The cylindrical SUS pipe 257 is designed to be longer in height than the piston lower member 256. The upper inner surface of the SUS pipe 257 is where the piston lower member 256 is inserted and coupled, and the lower part of the SUS pipe 257 is where the second movable body, which will be explained below, is inserted. In FIG. 11, the internal view of the SUS pipe 257 is shown, and FIG. 12 shows the internal view with the SUS pipe 257 removed.

Referring to FIGS. 13 to 15, the upper side of the second movable body 150 in the reusable module 100 has an upper coupling member 157, 158, which is inserted into the lower part of the SUS pipe 257. The upper coupling member is made of a metallic material with magnetic attraction properties, so when it is inserted into the lower part of the SUS pipe 257, the coupling magnet (M) and the upper coupling member are firmly connected by magnetic force. The upper coupling member 157, 158 may consist of a single metal part, but in this invention, it is composed of a central coupling member 157 located in the center and an outer coupling member 158 positioned on the outer side. When the reusable module and disposable module are coupled, it is preferable that the lower end of the SUS pipe is slightly inserted into the inside of the reusable module for secure coupling.

In the liquid infusion device of the present invention, the coupling of the second movable body 150 and the piston-check valve module 250 is essential for the piston operation when the reusable module 100 and disposable module 200 are connected. In this invention, a simple yet secure coupling is achieved by utilizing the coupling magnet (M) located inside the piston lower member 256 and the metallic upper coupling member 157, 158 provided on the top of the second movable body 150. As a result, when the solenoid 130 of the reusable module 100 operates and the first movable body 140 and second movable body 150 reciprocate up and down in sequence, this reciprocating motion is transmitted to the piston 255, allowing the liquid in the quantitative control space 251 to be discharged externally.

Additionally, the present invention includes a wake-up structure that supplies power to the device when the reusable module 100 and the disposable module 200 are connected. As a structure for the wake-up function, it is equipped with a wake-up coupling part 270 shown in FIG. 10 and a connection terminal part 170 shown in FIG. 13. When the reusable module 100 and the disposable module 200 are connected, the wake-up coupling part (270 in FIG. 10) and the connection terminal part (170 in FIG. 13) make contact. The wake-up coupling part 270 is equipped with a semicircular metal plate 275 inside, and the connection terminal part 170 contains three pogo pins 175. When the reusable module 100 and the disposable module 200 are coupled, the wake-up function is triggered by the electrical contact of two of the pogo pins 175 with the semicircular metal plate 275.

Additionally, the pogo pins 175 can also be used as charging pins. When the disposable module and the reusable module are separated, the pogo pins 175 can function as charging terminals to charge the battery built into the reusable module. In this case, the reusable module can be placed into a separate charging device designed for charging the reusable module, enabling it to enter a chargeable state.

Although the present disclosure has been described in connection with some examples herein, the present disclosure should not be limited to those examples only, and various other changes and modifications made by those skilled in the art from the basic concept of the disclosure are also within the scope of the claims appended herein.

What is claimed is:

1. A liquid infusion device comprising:
a reusable module (100) including a solenoid (130) that generates magnetic force when powered, a first movable body (140) that performs a reciprocating motion driven by the magnetic force generated by the solenoid, and a second movable body (150) that moves in conjunction with the reciprocating motion of the first movable body; and
a disposable module (200) including a reservoir (230) for storing the liquid and a piston-check valve module (250) that receives the liquid stored in the reservoir and discharges it externally;
wherein, when the reusable module and the disposable module are coupled, the piston-check valve module is connected to the second movable body through the attractive force of a magnet.

2. The liquid infusion device according to claim 1, further comprising a piston lower member (256), which is formed with an increased radius at the lower part of the piston (255); and a coupling magnet (M) provided in the piston lower member (256).

3. The liquid infusion device according to claim 2, wherein the upper side of the second movable body (150) is equipped with a top coupling member made of a metallic material that interacts with the magnetic force of the coupling magnet.

4. The liquid infusion device according to claim 3, wherein the top coupling member is composed of a central coupling member (157) positioned in the center and an outer coupling member (158) positioned on the outer side.

5. The liquid infusion device according to claim 4, wherein the disposable module is provided with a cylindrical SUS pipe (257) that surrounds and is coupled to the outer circumferential surface of the piston lower member (256), and
the upper inner surface of the SUS pipe (257) is where the piston lower member is inserted and coupled, and the lower part of the SUS pipe (257) is where the top coupling member is inserted and coupled to the coupling magnet (M).

6. The liquid infusion device according to claim 5, wherein, when the reusable module and the disposable module are coupled, the lower end of the SUS pipe is inserted into the inside of the reusable module for coupling.

7. The liquid infusion device according to claim 1, further comprising:
a semicircular metal plate (275) provided in the disposable module; and
a plurality of pogo pins (175) provided in the reusable module, positioned at the area where the metal plate makes contact;
wherein, when the reusable module and the disposable module are coupled, two of the pogo pins make electrical contact via the metal plate, thereby performing a wake-up function.

8. The liquid infusion device according to claim 7, wherein the pogo pins are used as charging terminals when the reusable module is in a separated state.
